(19) United States
(12) Patent Application Publication
(10) Pub. No.: US 2014/0303389 A1
(43) Pub. Date: Oct. 9, 2014

(54) PALM OIL ENRICHED IN UNSATURATED FATTY ACIDS

(71) Applicants: ARCHER DANIELS MIDLAND COMPANY, DECATUR, IL (US); NOVOZYMES A/S, BAGSVAERD (DK); FRITO - LAY NORTH AMERICA, INC., PLANO, TX (US)

(72) Inventors: Thomas George Crosby, Frisco, TX (US); Marianne Linde Damstrup, Dentofte (DK); John Inmok Lee, Decatur, IL (US); Per Munk Nielson, Hillerod (DK); Craig Jordan Weitz, Dallas, TX (US)

(21) Appl. No.: 14/358,349

(22) PCT Filed: Nov. 20, 2012
(86) PCT No.: PCT/US12/66032

§ 371 Date: May 15, 2014

Related U.S. Application Data
(60) Provisional application No. 61/562,501, filed on Nov. 22, 2011.

(51) Int. Cl. C12P 7/64

(52) U.S. Cl. CPC C12P 7/6454
USPC 554/227; 435/134

(57) ABSTRACT

A process for producing a food oil containing at least 50% monounsaturated fatty acids from palm oil is disclosed. Fatty acids are released from palm oil glycerides, such as by fat-splitting. The free fatty acids (FFA) are separated to obtain a fraction enriched in unsaturated palm fatty acid. This fraction is subject to a condensation reaction with glycerol to form an oil comprising mainly triglycerides (triacylglycerols). The condensation reaction is catalyzed by an enzyme.

### FIELD OF THE INVENTION

The present invention relates to processes for producing a palm oil substitute enriched in unsaturated fatty acids as well as to palm oil substitutes produced by the processes.

### BACKGROUND OF THE INVENTION

Palm oil is abundant and widespread in tropical regions. Residents of tropical countries have ready access to palm oil for cooking and frying. However, palm oil is problematically high in saturated fats, especially palmitic acid. Palmitic acid is a type of saturated fat that reportedly has adverse effects on cholesterol levels. In addition, according to the World Health Organization, the palmitic acid in palm oil is linked to heart disease. The American Diabetes Association recommends eating less saturated fats, which includes palm oil. However, tropical countries often do not support the production of oilseeds that are lower in saturated fats, and thus higher in unsaturated oils, than palm oil. Because palm oil trees produce more oil per hectare than most other food oil crops, palm oil production is a more efficient use of the land area than other oilseeds to provide needed calories. In transportation, unsaturated oils may be subjected to oxidative stresses, such as heat, metal in transport vessels, and water. Unfortunately, the unsaturated oils are more susceptible to oxidation than saturated fats, causing oxidative breakdown in the quality of the unsaturated oils in transit. Oxidation, in turn, leads to development of rancidity, off-flavors, and, in extreme cases, renders the oil unsuitable for consumption. For these reasons, tropical diets are often deficient in healthy unsaturated oils and supply an excess of less desirable saturated fats.

To address this problem, we have developed a novel process that employs palm oil itself as a source of unsaturated fatty acids. Palm oil is disassembled into component fatty acids. Saturated fatty acids, such as lauric acid, myristic acid, and palmitic acid are removed, and the oil is reassembled. An oil sourced entirely from palm is obtained; the oil is enriched in desirable unsaturated fatty acids from palm oil. The oil is suitable for frying and incorporating into edible products.

Frying is widely used in food preparation worldwide. Frying food improves taste, color, and the shelf-life of food. The fat or oil used to fry food acts as a heat transfer medium and is taken up by the food, enhancing the flavor and mouthfeel. Frying oil comes into close contact with exterior food surfaces, facilitating even transformation of the food.

One of the most important characteristics of frying oil is the ability of the oil to operate at the high temperatures needed for frying. Other important factors include resistance to foaming, darkening, smoking, and gum formation, and a low rate of development of free fatty acids. Frying usually is carried out at 160-190° C.

One approach the food industry has applied to improving the oxidative stability of oils is hydrogenation, also known as hardening. In hydrogenation, some or all unsaturated bonds are chemically reduced to saturated bonds and the oil is partially or completely converted to saturated fats. However, this is accompanied by certain drawbacks; the melting points of saturated fatty acids are higher than the melting points of the corresponding original unsaturated fatty acids and substantially higher than the melting points of the corresponding original polyunsaturated fatty acids. Most hardened fats have melting points above mouth temperature, rendering them unsuitable for frying food due to a waxy mouthfeel inconsistent with food products. Alternatively, partial hydrogenation does not result in the same amount of melting point elevation, but results in the development of trans-unsaturated fatty acids. Trans fatty acids are widely recognized as unhealthy and are subject to labeling requirements. Moreover, consumers are increasingly averse to buying food containing trans fats or food containing the word “hydrogenated” on the label.

A solution to the long-felt need for food oils having lower saturated fatty acid content and enriched in oleic acid content is genetic modification of oilseed germplasm, and significant effort has been expended to genetically modify oilseed traits to produce high oleic oils. High-oleic sunflower oil, high-oleic soybean oil and even high-oleic palm oil have been generated; however, the use of oils from genetically modified sources is repugnant in some regions.

As an alternative to hydrogenation and genetic modification, the food industry has developed methods of changing the chemical structure of oils and fats by replacing fatty acid components of the triacylglycerols of oil and fats. These processes employ catalysts to carry out ester-exchange, or interesterification, reactions to create so-called “structured lipids.” These can be distinguished from simple blends of the same oils by analysis, and the physical properties of structured lipids are usually different from the physical properties of simple blends of the same oils.

Historically, chemical catalysts such as sodium hydroxide or sodium methoxide have been used in industrial-scale reactors to form structured lipids. However, these catalysts are accompanied by oil oxidation and the formation of soaps and free fatty acid by-products, which cause a significant loss of oil product yield and additional effort and cost to remove them. The uncontrolled nature of the chemical catalyst results in a random distribution of fatty acids across the glycerol backbone of a fat or oil, which can negatively affect the properties of the product. To overcome these drawbacks, enzymes catalysts are employed. Enzymes typically operate at lower temperatures than chemical catalysts, which has the advantage of decreasing the danger of oxidation when working with fats and oils. Due to their selective nature, enzyme catalysts may result in fewer by-product losses, and a desired non-random distribution of fatty acids in a fat or oil can be achieved by proper selection of enzyme and operating conditions.

In the condensation of glycerol and free fatty acids to form triacylglycerols (TAG), the reaction intermediates diacylglycerol (DAG) and monoacylgycerol (MAG) are formed. These reaction intermediates (DAG and MAG) are referred to as “partial glycerides.” In practical use, triacylglycerol oils having levels of monoacylglycerols above about 1 weight percent may be prone to difficulties in the purification steps of physical refining or deodorization, and in frying, especially in industrial fryers. Smoking may result due to depressed oil flash point or a decreased smoke point due to the presence of monoacylglycerols. In addition, diacylglycerols are often unwanted byproducts in triacylglycerol oil due to the presence of free hydroxyl groups and resulting lack of stability of the oil.

Free fatty acids should be limited because of their susceptibility to oxidation as well as of their contribution to smoke when heating at frying temperatures.

Condensation reactions between an acid and alcohol to form an ester can be accelerated by a broad range of catalysts and catalytic effects, such as heat, acid, and alkali. Employing these catalysts and catalytic effects may be accompanied by formation of unwanted by-products, necessitating costly and potentially cumbersome purification steps. The use of biological catalysts, such as lipases (triacylglycerol acylhydrolases EC 3.1.1.3) enables processing under mild conditions and generate fewer by-products than non-biological catalysts.

Frying oils are prone to oxidative decomposition in use. The products of oxidative decomposition reduce the useful life of the oil. Palm oil is a preferred frying oil because of superior resistance to oxidation when exposed to heat and the moisture released from frying food. Palm oil is used worldwide as a frying oil in restaurants, in fast food outlets, in large-scale par-frying, in snack food preparation, and in the preparation of instant noodles. Palm oil is easily filtered and has an unusually complex group of antioxidants such as tocols (tocopherols and tocotrienols), sterols, aliphatic triterpenes (squalene), ubiquinone, and carotenoids. The broad range of antioxidants in palm oil promotes resistance to oxidation.

Palm oil contains primarily palmitic acid (a saturated fatty acid having 16 carbons in a chain configuration, about 44%) and oleic acid (a monounsaturated fatty acid having eighteen carbons in a chain configuration, about 39%). Oleic acid has a single site of unsaturation and is therefore more heat stable than the polyunsaturated fatty acids abundant in non-tropical oils. However, palm oil is noted for having poor cold stability; that is, when cooled or refrigerated, palm oil crystallizes and hardens to a solid due to the high content of saturated fatty acids present. This makes the palm oil more difficult to use. In addition, margarine manufacture from palm oil is difficult due to the long crystallization times of palm oil.

Crude palm oil as a starting material for edible oil is traded under the trading rules of the National Institute of Oilseed Products, which specify that crude palm oil must contain at most 5% free fatty acids and at most 1% moisture and impurities. In addition, crude palm oil typically contains levels of diacylglycerols (DAG) and free fatty acids (FFA) on the order of several percent (Table A).

TABLE A
Composition of crude palm oil from fresh ripe fruit and average
composition of traded palm oil. From Bailey's Industrial Oil and
Fat Products, Volumes 1-6 (6th Edition), Vol. 2, Chapter 8; Yusof
Basiron, Palm Oil, pp. 333-430; Edited by: Shahidi, Fereidoon ©
2005 John Wiley & Sons; Hoboken, NJ, USA; table 37 from page 382.

| Component | Ripe, Fresh, Unbruised Fruit | Average Traded PO |
|---|---|---|
| Triglycerides (%) | 98 | <98 |
| Diglycerides (%) | 2-3 | 4-8 |
| Monoglycerides (%) | 0.1 | 0.2 |
| FFA (%, as C16:0) | 0.1 | 3.5 (max 5) |
| Phosphorus (ppm) | 2-3 | 20-30 |
| Tocopherols (ppm) | 800 | 600-800 |
| Carotene (ppm) | 550 | 550 |
| Totox | 1 | >5 |
| Iron (mg/kg) | 0.1-0.3 | 5-10 |
| Copper (mg/kg) | 0.01 | 0.05 |

It would be desirable to produce an enzymatically condensed or interesterified palm oil having a content of unsaturated fatty acids greater than 70 wt. % that is similar in triacylglycerol, diacylglycerol, monoacylglycerol, and free fatty acid content to average traded crude palm oil without additional processing steps.

There is a long-felt need in the food industry for a frying oil with good oxidative stability, but that does not contain hydrogenated oil or oil from genetically modified plants. The present invention provides stable frying oils, as well as methods of making them, from fatty acids sourced from palm oil.

### SUMMARY OF THE INVENTION

A non-limiting aspect of the present disclosure is directed to a process for producing a palm oil substitute triacylglycerol product enriched in monounsaturated fatty acids comprising the steps of providing a palm free fatty acid feedstock comprising saturated and monounsaturated fatty acids, subjecting the feedstock to separation to obtain a fraction enriched in free monounsaturated fatty acids and a fraction enriched in saturated fatty acids, and esterifying glycerol and the fraction enriched in free monounsaturated fatty acids to produce a condensation triacylglycerol oil comprising a palm oil substitute triacylglycerol product enriched in monounsaturated fatty acids, wherein the esterification step is performed by contacting the glycerol and the fraction enriched in free monounsaturated fatty acids with one or more enzymes under conditions comprising that water formed in the condensation reaction is eliminated from the reaction mixture, and a temperature of at least 70° C. In certain non-limiting aspects of the process, the esterification step is performed under conditions comprising a water content of less than or equal to 700 ppm, 650 ppm, less than or equal to 600 ppm, less than or equal to 550 ppm, or less than or equal to 500 ppm. In still other non-limiting aspects, the esterification step is performed under conditions comprising a pressure of less than 40 kPa, less than 30 kPa, less than 20 kPa, less than 15 kPa, less than 10 kPa, less than 5 kPa, less than 4 kPa, less than 3 kPa, less than 2 kPa, or less than 1 kPa. In additional non-limiting aspects of the process, the esterification step is performed under conditions comprising a temperature of at least 75° C., at least 80° C., at least 81° C., or at least 82° C. In still further non-limiting aspects of the process, the free fatty acid feedstock is derived from one or more oil selected from the group consisting of palm oil, palm-kernel oil, crude palm oil, refined palm oil, physically refined palm oil, deodorized palm oil, palm fractions, palm olein, palm stearin, palm mid fraction, and combinations of any thereof. In alternative non-limiting aspects of the process, the fatty acid feedstock comprises one or more oil selected from the group consisting of palm sludge oil, palm oil mill effluent, palm fatty acid distillate, and combinations of any thereof. In yet other non-limiting embodiments of the process, the free fatty acid feedstock is derived from fat splitting of one or more acylglycerols. In another non-limiting aspect, the free fatty acid feedstock is derived from enzymatic hydrolysis of one or more triacylglycerols. In alternative non-limiting aspects, separating the palm free fatty acid feedstock is conducted with a process selected from the group consisting of: distillation, crystallization, centrifugation, urea precipitation, membrane filtration; molecular sieve, directed interesterification; and any combination thereof. In further non-limiting aspects, the fraction enriched in free monounsaturated fatty acids comprises at least 50 wt. %, at least 60 wt. %, at least 70 wt. %, at least 80 wt. %, at least 85 wt. %, or at least 90 wt. % monounsaturated fatty acids. In still further non-limiting aspects of the process, the esterification in step c) is catalyzed by an immobilized lipase, preferably a lipase having at least 70%, at least 80%, at least 90%, at least 95%, at least 96%, at least 97%, at least 98%, or at least 99% sequence identity to the sequence shown in SEQ ID NO:1 of PCT patent application WO2008065060. In further non-limiting aspects of the process, the fraction enriched in saturated fat fraction comprises at least 65 wt. % saturated fatty acids and at least 60 wt. % palmitic acid.

In further non-limiting aspects of the process, the palm oil substitute triacylglycerol product following the esterification step has a content of monoacylglycerols less than or equal to 3 wt. %, less than or equal to 2 wt. %, less than or equal to 1.5 wt. %, less than or equal to 1 wt. %, or less than or equal to 0.5 wt. %. In additional non-limiting aspects of the process, the palm oil substitute triacylglycerol product following the esterification step has a content of diacylglycerols less than or equal to 8 wt. %, less than or equal to 7 wt. %, less than or equal to 6 wt. %, less than or equal to 5 wt. %, less than or equal to 4 wt. %, less than or equal to 3 wt. %, less than or equal to 2 wt. %, or less than or equal to 1 wt. %. In certain additional non-limiting aspects of the process, the palm oil substitute triacylglycerol product following the esterification step has a content of free fatty acids less than or equal to 3 wt. %, less than or equal to 2.5 wt. %, less than or equal to 2 wt. %, less than or equal to 1.5 wt. %, less than or equal to 1 wt. %, or less than or equal to 0.5 wt. %. In still further non-limiting aspects of the process, the palm oil substitute triacylglycerol product comprises at least 90 wt %, at least 91 wt. %, at least 92 wt %, at least 93 wt. %, at least 94 wt. %, at least 95 wt. %, at least 96 wt. %, at least 97 wt. %, or least 98 wt. % triacylglycerol. In certain non-limiting aspects of the process, the palm oil substitute triacylglycerol product comprises at least 70 wt. %, at least 80 wt. %, at least 85 wt. %, at least 90 wt. %, or at least 95 wt. % unsaturated fatty acids. In still further non-limiting aspects of the process, the palm oil substitute triacylglycerol product is subjected to deodorization. In further non-limiting aspects of the process, the palm oil substitute triacylglycerol product enriched in monounsaturated fatty acids comprises 90 to 98 wt. % triacylglycerol, 4 to 8 wt. % diacylglycerol, a maximum of 0.2 wt. % monoacylglycerol, and a maximum of 5 wt. % free fatty acid. In a further non-limiting aspect of the process, compositions produced by any of the preceding processes are disclosed.

### DETAILED DESCRIPTION OF THE INVENTION

As used herein, “condensation”, “esterification” or “ester synthesis” means the reaction of an alcohol with an acid, especially a free fatty acid, leading to formation of an ester. During the condensation reactions described in this application, free fatty acids present in starting materials may react with polyhydric alcohols, such as glycerol or monoacylglycerols, or with monohydric alcohols, such as diacylglycerols. As used herein, “interesterification” reactions mean the following reactions: acidolysis, transesterification, ester exchange, and alcoholysis, as indicated in Formo, M., J. Amer. Oil Chem. Soc. 31, 548-559 (1954).

As used herein, “lipase-catalyzed reactions,” “contacting an oil with an enzyme,” and “incubating an oil with an enzyme” each mean the following reactions: hydrolysis, esterification, transesterification, acidolysis, interesterification, and alcoholysis. As used herein, “acylglycerols” means glycerol esters commonly found in oil, such as monoacylglycerols, diacylglycerols, and triacylglycerols.

As used herein, “lipase” means triacylglycerol acylhydrolase (EC 3.1.1.3) and includes enzymes that facilitate condensation (ester synthesis) reactions, alcoholysis reactions, acidolysis reactions, and interesterification (ester exchange or transesterification) reactions.

As used herein, “average traded crude palm oil” means a palm oil comprising about 3.5 wt. % free fatty acids and at most 5 wt. % free fatty acids; at most 0.2 wt. % monoacylglycerols; 4-8 wt. % diacylglycerols, and less than 98 wt. % triacylglycerols, as described in Table A herein (Bailey's Industrial Oil and Fat Products, Fifth Edition, Y. H. Hui, Ed., 2005, Volume 2, Chapter 8, Palm Oil, Y. Basiron, Table 37. Crude Palm Oil Quality, p. 382). In one embodiment, the average traded crude palm oil comprises at least 90 wt. % triacylglycerols.

As used herein “palm oil substitute enriched in monounsaturated fatty acids” means an oil comprising predominantly triacylglycerols, wherein fatty acid constituents of the oil are sourced from palm oil, and the content of unsaturated fatty acids in the palm oil substitute is greater than the content of unsaturated fatty acids in the source palm oil. The palm oil substitute enriched in free monounsaturated fatty acids is suitable for substantially the same uses as a traded crude palm oil: however, the palm oil substitute is enriched in monounsaturated fatty acids and comprises a lower content of palmitic acid than average traded crude palm oil

As used herein “fraction enriched in free monounsaturated fatty acids” means a fraction resulting from a separation process wherein palm oil, palm oil acylglycerols, palm oil free fatty acids, or combinations of any thereof is separated into two or more fractions based on degree of unsaturation; the content of unsaturated fatty acids in the fraction enriched in free monounsaturated fatty acids is greater than the content of unsaturated fatty acids in the palm free fatty acid feedstock.

As used herein, “frying stable oil” means an oil produced by the process described herein suitable for typical frying operations. In one embodiment, the stable frying oil starts with less than 2 wt. % omega-3 fatty acids, a free fatty acid content of less than 0.05 wt. %, peroxide value of <1 meq O2/kg, and have oxidative stability sufficient to provide fried food products with satisfactory taste and shelf life. Oil may be supplemented with antioxidant to provide an oil having an oxidative stability of greater than 20 hours measured by OSI at 110° C. A high smoke point is also desirable.

As used herein, “palm free fatty acid feedstock” means a feedstock comprising fatty acids originating from the fruit of palm trees, such as Elaies guineensis and Elaies oleifera. Palm free fatty acid feedstock includes fatty acids from palm oil, crude palm oil, refined palm oil, physically refined palm oil, deodorized palm oil, palm oleins, palm stearins, palm mid fractions, palm sludge oil, palm oil mill effluent, and palm fatty acid distillate (PFAD). Combinations of palm free fatty acid feedstocks can be used. The free fatty acids can be derived from palm oil acylglycerols, such as triacylglycerols, diacylglycerols, monoacylglycerols, and from esters, such as waxes; the fatty acids can alternatively be derived from free fatty acid streams. Free fatty acid streams are not readily available in nature, but are obtained by hydrolysis (splitting) of acylglycerols, primarily hydrolysis of triacylglycerols. For example, palm oil often contains significant amounts of free fatty acids (3.5 wt. %) resulting from hydrolysis of palm acylglycerols; the free fatty acids are removed from the oil and concentrated in palm fatty acid distillate. Combinations of these may be used, as well as combinations of palm fatty acid feed stock and fatty acid feed stock from other vegetable sources. As used herein, “comprising saturated and monounsaturated fatty acids” means that the palm free fatty acid feedstock comprises both saturated fatty acids, such as palmitic acid or stearic acid, and unsaturated fatty acids, such as oleic acid or linoleic acid.

In addition to fatty acids originating from palm the “palm free fatty acid feedstock” may comprise from other vegetable sources. Such other vegetable sources acids include fatty acids originating from vegetable sources oils similar to palm oil, i.e., are high in saturated fatty acid and low in monounsaturated fatty acid, such as rice bran oil, animal fat, including milk fat, coconut oil, palm kernel oil, cocoa butter, avocado oil, and nut oils, such as pili nuts and cottonseed oil.

The palm free fatty acid feed stock may be provided by fat splitting of acylglycerols by any of the widely-practiced methods or improvements. Agitating fat with water under pressure and at high temperatures in the presence of a catalyst is a general approach for production of free fatty acids. If an alkali is included in the hydrolysis, saponification takes place, in which the free fatty acids form soaps or salts with the alkali material. Treatment with acid is sufficient to release the free fatty acids from the soaps. Conventional fat splitting processes include the Twitchell process, the Autoclave process, the Colgate-Emory process, Saponification, and enzymatic de-esterification (hydrolysis) of palm fatty acid feedstock. Enzymatic de-esterification of palm fatty acid feedstock may be performed using a lipase (EC 3.1.1.3). Alternatively, fat may be agitated with water under pressure at a maximum temperature of 250° C. in the absence of a catalyst to provide free fatty acids; this approach minimizes the formation of trans fatty acids.

According to the invention the palm free fatty acid feedstock is subjected to separation to obtain a fraction enriched in free monounsaturated fatty acids. Suitable methods include distillation, in which palmitic acid is removed in the distillate and unsaturated fatty acids are enriched in the undistilled residue (bottoms). Chromatography, such as argentation chromatography or other complexation chromatography is suitable. In an aspect, crystallization, such as dry fractionation or solvent fractionation may be selected. Urea complexation, in which saturated fatty acids form inclusion complexes which are separated as precipitates is also suitable. In another aspect, centrifugation may be carried out, and may be combined with crystallization or urea complexation. In addition, membrane filtration may be suitable for separation; membrane filtration may be combined with crystallization or urea complexation. In an aspect, molecular sieves, such as a crystalline silica having a silica/alumina molar ratio ≧12, may effect separation to obtain a faction enriched in free monounsaturated fatty acids. In another aspect, directed interesterification may be carried out, in which esters of saturated fatty acids are formed and precipitate from the reaction solution. After enrichment of an ester feedstock enriched in saturated fatty acids, the desired fraction may be converted to free fatty acids In another aspect, the palm free fatty acid feedstock may be converted to esters, such as methyl esters, and separated by any of the foregoing methods. After enrichment of an ester feedstock enriched in unsaturated fatty acids, the desired fraction may be converted to free fatty acids. Any combination of the foregoing methods may be used.

The fraction enriched in free monounsaturated fatty acids comprises at least 50 wt. %, at least 60 wt. %, at least 70 wt. %, at least 80 wt. %, or even at least 90 wt. % monounsaturated fatty acids.

According to the invention glycerol and the palm free fatty acid fraction enriched in free monounsaturated fatty acids are esterified to produce a condensation triacylglycerol oil comprising a palm oil substitute triacylglycerol product similar in TAG, DAG, MAG and FFA content to average traded crude palm oil and enriched in monounsaturated fatty acids. The esterification is performed by contacting the glycerol and a fraction enriched in free monounsaturated fatty acids with one or more lipase enzymes.

Sufficient agitation should be carried out to ensure that mass transfer is not rate-limiting in the condensation reaction, and can be carried out by any of a number of widely practiced methods, such as stirring, swirling, gas bubbling, ultrasound, and flow velocity.

During the esterification the water formed in the condensation reaction is eliminated from the reaction mixture. The equilibrium of the reaction is thereby pushed towards esterification. Furthermore, performing the esterification under conditions comprising elimination of water formed in the condensation reaction from the reaction mixture and maintaining a temperature of at least 70° C. enables achieving a of very low content of mono- and diacylglycerols as well as of FFA.

Preferably the palm oil substitute triacylglycerol product comprises at least 90 wt. %, at least 91 wt. %, at least 92 wt. %, at least 93 wt. %, at least 94 wt. %, at least 95 wt. %, at least 96 wt. %, at least 97 wt. %, or least 98 wt. % triacylglycerol.

The palm oil substitute triacylglycerol product may comprise at least 75 wt. %, at least 80 wt. %, at least 85 wt. %, at least 90 wt. %, or at least 95 wt. % unsaturated fatty acids reflecting the composition of the fraction enriched in free monounsaturated fatty acids applied in the condensation reaction.

Preferably the palm oil substitute triacylglycerol product following step (c) has a content of free fatty acids less than or equal to 3 wt. %, less than or equal to 2.5 wt. %, less than or equal to 2 wt. %, less than or equal to 1.5 wt. %, less than or equal to 1 wt. %, or less than or equal to 0.5 wt. %.

Preferably the palm oil substitute triacylglycerol product following step (c) has a content of monoacylglycerols less than or equal to 3 wt. %, less than or equal to 2 wt. %, less than or equal to 1.5 wt. %, or less than or equal to 1 wt. %, or less than or equal to 0.5 wt. %.

Preferably the palm oil substitute triacylglycerol product following step (c) has a content of diacylglycerols less than or equal to 8 wt. %, less than or equal to 7 wt. %, less than or equal to 6 wt. %, less than or equal to 5 wt. %, less than or equal to 4 wt. %, less than or equal to 3 wt. %, less than or equal to 2 wt. %, or less than or equal to 1 wt. %.

Preferably the water content of the reaction mixture is less than or equal to 700 ppm, 650 ppm, less than or equal to 600 ppm, less than or equal to 550 ppm, or even less than or equal to 500 ppm.

Water removal from a reaction mixture may be accomplished by several methods, including operating the esterification reaction under full or partial vacuum; contacting the reaction mixture or the headspace of the reaction mixture with a drying adsorbent, such as molecular sieves; contacting the reaction mixture or the headspace of the reaction mixture with dry gas, such as nitrogen or carbon dioxide; contacting the headspace of the reaction mixture with a cold surface onto which water of reaction will condense as ice; and spraying the reaction mixture into a vessel under vacuum. Combinations of these methods can be used.

The esterification may be performed under conditions comprising reduced pressure, e.g., a pressure of less than or equal to 40 kPa, less than or equal to 30 kPa, less than or equal to 20 kPa, less than or equal to 15 kPa, less than or equal to 10 kPa, less than or equal to 5 kPa, less than or equal to 4 kPa, less than or equal to 3 kPa, less than or equal to 2 kPa, less than or equal to 1 kPa.

The esterification is performed under conditions comprising a temperature of at least 70° C., such as at least 75° C., at least 80° C., at least 81° C., or at least 82° C.

In an embodiment the process step b) further comprises subjecting the feedstock to separation to obtain a fraction enriched in free saturated fatty acids. The fraction enriched in saturated fat fraction comprises preferably at least 60 wt. %, at least 70 wt. %, at least 80 wt. %, or even at least 90 wt. % saturated fatty acids. This fraction and an alcohol, preferably methanol or ethanol, may be subjected to esterification comprising contacted with one or more lipase enzymes, to produce fatty acid alkyl esters. The fatty acid alkyl esters may be used as biodiesel. Alternatively, the fraction enriched in free saturated fatty acids may be subjected to esterification to provide a highly saturated triacylglycerol oil.

A suitable lipase enzyme for use in the present invention may be a lipase belonging to EC 3.1.1.3, e.g., one selected from group consisting of the Candida antarctica lipase A (CALA) as disclosed in WO 88/02775, the C. antarctica lipase B (CALB) as disclosed in WO 88/02775 and shown in SEQ ID NO:1 of WO2008065060, the Thermomyces lanuginosus (previously Humicola lanuginosus) lipase disclosed in European patent application EP 258 068), the Thermomyces lanuginosus variants disclosed in PCT patent application WO 2000/60063 or WO 1995/22615, in particular the lipase shown in positions 1-269 of SEQ ID NO: 2 of WO 95/22615, the Hyphozyma sp. lipase (WO 98/018912), and the Rhizomucor miehei lipase (SEQ ID NO:5 in WO 2004/099400), a lipase from P. alcaligenes or P. pseudoalcaligenes (EP 218 272), P. P. cepacia (EP 331 376), P. glumae, P. stutzeri (GB 1,372,034), P. fluorescens, Pseudomonas sp. strain SD 705 (PCT patent application WO 95/06720 and WO 96/27002), P. wisconsinensis (WO 96/12012); a Bacillus lipase, e.g., from B. subtilis (Dartois et al. (1993), Biochemica et Biophysica Acta, 1131, 253-360), B. stearothermophius (Japanese patent application JP 64/744992) or B. pumilus (WO 91/16422). Also suitable is a lipase from any of the following organisms: Fusarium oxysporum, Absidia reflexa, Absidia corymbefera, Rhizomucor miehei, Rhizopus delemar (oryzae), Aspergillus niger, Aspergillus tubingensis, Fusarium heterosporum, Aspergillus oryzae, Penicilium camembertii, Aspergillus foetidus, Aspergillus niger, Aspergillus oryzae and Thermomyces lanuginosus, such as a lipase selected from any of SEQ ID NOs: 1 to 15 in PCT patent application WO 2004/099400.

A preferred lipase enzyme for the invention is the C. antarctica lipase B (CALB) as disclosed in PCT patent application WO 88/02775 and having the sequence shown in SEQ ID NO:1 of WO2008065060. Also preferred are lipase enzymes having at least 70%, at least 80%, at least 90%, at least 95%, at least 96%, at least 97%, at least 98%, or even at least 99% sequence identity to the sequence shown in SEQ ID NO:1 of PCT patent application WO2008065060.

The lipase enzyme used in the process of the invention may be derived or obtainable from any of the sources mentioned herein. The term “derived” means in this context that the enzyme may have been isolated from an organism where it is present natively, i.e. the identity of the amino acid sequence of the enzyme are identical to a native enzyme. The term “derived” also means that the enzymes may have been produced recombinantly in a host organism, the recombinant produced enzyme having either an identity identical to a native enzyme or having a modified amino acid sequence, e.g. having one or more amino acids which are deleted, inserted and/or substituted, i.e. a recombinantly produced enzyme which is a mutant and/or a fragment of a native amino acid sequence. Within the meaning of a native enzyme are included natural variants. Furthermore, the term “derived” includes enzymes produced synthetically by e.g. peptide synthesis. The term “derived” also encompasses enzymes which have been modified e.g. by glycosylation, phosphorylation etc., whether in vivo or in vitro. The term “obtainable” in this context means that the enzyme has an amino acid sequence identical to a native enzyme. The term encompasses an enzyme that has been isolated from an organism where it is present natively, or one in which it has been expressed recombinantly in the same type of organism or another, or enzymes produced synthetically by e.g. peptide synthesis. With respect to recombinantly produced enzyme the terms “obtainable” and “derived” refers to the identity of the enzyme and not the identity of the host organism in which it is produced recombinantly.

Accordingly, the lipase enzyme may be obtained from a microorganism by use of any suitable technique. For instance, an enzyme preparation may be obtained by fermentation of a suitable microorganism and subsequent isolation of an enzyme preparation from the resulting fermented broth or microorganism by methods known in the art. The enzyme may also be obtained by use of recombinant DNA techniques. Such method normally comprises cultivation of a host cell transformed with a recombinant DNA vector comprising a DNA sequence encoding the enzyme in question and the DNA sequence being operationally linked with an appropriate expression signal such that it is capable of expressing the enzyme in a culture medium under conditions permitting the expression of the enzyme and recovering the enzyme from the culture. The DNA sequence may also be incorporated into the genome of the host cell. The DNA sequence may be of genomic, cDNA or synthetic origin or any combinations of these, and may be isolated or synthesized in accordance with methods known in the art.

The optimum parameters for enzymatic activity will vary depending upon the enzyme used. The rate of enzyme degradation depends upon factors known in the art, including the enzyme concentration, substrate concentration, temperature, the presence or absence of inhibitors and presence of water. These parameters may be adjusted to optimise the esterification reaction.

An enzyme composition immobilized on a hydrophobic carrier is preferred for the invention. The use of immobilized enzymes in processing of oils has experienced significant growth due to new technology developments that have enabled cost effective methods. A fundamental advantage of immobilized enzymes is that they can be recovered and re-used from a batch process by simple filtration or fixed in a column for continuous use.

Various ways of immobilizing lipase enzymes are well known in the art. A review of lipase immobilization is found in “Immobilized lipase reactors for modification of fats and oils—a review” Malcata, F X., et al. (1990) J. Amer. Oil Chem. Soc. Vol. 67 p. 890-910, where examples of representative lipase immobilizing carriers are illustrated, including inorganic carriers such as diatomaceous earth, silica, porous glass, etc.; various synthetic resins and synthetic resin ion exchangers; and natural polysaccharide carriers such as cellulose and cross-linked dextrin introduced with ion exchange groups.

In some embodiments the invention relates to a method, wherein the lipase enzyme is immobilized either on a carrier; by entrapment in natural or synthetic matrices, such as sol-gels, alginate, and carrageenan; by cross-linking methods such as in cross-linked enzyme crystals (CLEC) and cross-linked enzyme aggregates (CLEA); or by precipitation on salt crystals such as protein-coated micro-crystals (PCMC).

In some embodiments the invention relates to a method, wherein the carrier is a hydrophilic carrier selected from the group containing: porous inorganic particles composed of alumina, silica or silicates such as porous glass, zeolites, diatomaceous earth, bentonite, vermiculite, hydrotalcite; and porous organic particles composed of carbohydrate polymers such as agarose or cellulose.

In some embodiments the invention relates to a method, wherein the carrier is a hydrophobic carrier selected from the group containing: synthetic polymers such as nylon, polyethylene, polypropylene, polymethacrylate, or polystyrene; and activated carbon. Suitable commercial carriers are e.g., LEWATIT™, ACCUREL™, PUROLITE™, DUOLITE™ and AMBERLITE™.

Suitable commercial enzyme compositions include LIPOZYME RM IM™ comprising an immobilized lipase from Rhizomucor miehei as well as LIPOZYME® 435 and NOVOZYM® 435, both comprising an immobilized lipase B from Candida antarctica.

Typically, the enzyme is used in a concentration corresponding to 1 PLU/g fatty acid feedstock to 1000 PLU/g fatty acid feedstock. Preferably the enzyme is used in a concentration of between 5 PLU/g fatty acid feedstock to 500 PLU/g fatty acid feedstock, more preferably between 10 PLU/g fatty acid feedstock to 100 PLU/g fatty acid feedstock. The PLU is defined below.

The process design applied for performing the esterification may be selected from the group of consisting of: batch, continuous stirred tank reactor, packed bed reactor, moving packed bed reactor, and expanded bed reactor.

Sequence identity. The identity of an enzyme may be calculated based on either amino acid sequences or nucleotide sequences. The relatedness between two amino acid sequences or between two nucleotide sequences is described by the parameter “identity”. For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is designated the percent identity and is calculated as follows:

Percent identity=(Identical Residues×100)/(Length of Alignment−Total Number of Gaps in Alignment)

In a preferred embodiment the palm oil substitute triacylglycerol product enriched in monounsaturated fatty acids is subjected to deodorization. The deodorization comprises distillation of a triacylglycerol oil, with optional steam sparging, to remove free fatty acids and any volatile material in the oil, such as flavors, odors, and oxidation products. Oil is heated, often to over 100° C., under vacuum and water or steam is introduced into the oil. Unwanted volatiles are released from the oil and carried away into a deodorizer distillate fraction. Palm oil is often processed by so-called “physical refining,” which is similar to deodorization and comprises high temperature distillation of oil under conditions which remove most free fatty acids while keeping the bulk of triacylglycerols intact. Deodorization is preferably carried out under conditions which minimize isomerization of fatty acids and components of fats and oils to trans isomers.

The process disclosed is equally applicable to other oils that have a fatty acid composition similar to palm oil, i.e., are high in saturated fatty acid and low in monounsaturated fatty acid, such as rice bran oil and cottonseed oil.

The following examples illustrate methods for providing palm oil substitute triacylglycerol product enriched in monounsaturated fatty acids, and compositions of oils similar in TAG, DAG, MAG and FFA content to average traded crude palm oil, according to the present invention. The following examples are illustrative only and are not intended to limit the scope of the invention as defined by the appended claims.

### Methods and Materials

The activity of immobilised lipases may be determined as Propyl Laurate Units (PLU/g). The immobilised lipase esterifies lauric acid with 1-propanol, forming propyl laurate. The activity (pmol/g/min) is determined by quantification of formed propyl laurate and consumed lauric acid by GC. Reaction temperature is 60° C. and reaction time 20 min. One PLU unit corresponds to 1 μmol/g/min, e.g. 1 μmol propyl laurate formed per g of enzyme product per minute.

Enzyme LIPOZYME® 435 is a commercial enzyme product from Novozymes A/S comprising an immobilized lipase B from Candida antarctica having the sequence shown in SEQ ID NO:1 of WO2008065060. The product has an activity of 10000 PLU/g. LIPOZYME® 435 is available from Novozymes A/S. Novozymes NS-40083 lipase is an immobilized lipase B from Candida antarctica having the sequence shown in SEQ ID NO:1 of WO2008065060.

All reagents used were at least technical grade. Quantitation of triacylglycerols, diacylglycerols, monoacylglycerols, and free fatty acids was carried out by gas chromatography after derivatization or High performance Liquid Chromatography-Size Exclusion Chromatography (HPLC-SEC).

### EXAMPLES

### Example 1

### Esterification of Glycerol and Free Fatty Acids to Form Triacylglycerols

An esterification reaction of free fatty acid with glycerol was carried out. In an esterification (condensation) reaction, stoichiometric amounts of glycerol and free fatty acids were incubated with a lipase. Linoleic acid (118 grams) was stirred with LIPOZYME® 435 lipase (18.8 grams) for ten minutes at 50° C., then glycerol (20.4 grams) was added and the reaction mixture was stirred at 450 rpm and 70° C. for 22 hours under reduced pressure (0.66 kPa) to eliminate water formed in the condensation reaction from the reaction mixture, Results are shown in Table 1.

TABLE 1

| | Composition (wt. %) | | | | |
|---|---|---|---|---|---|
| Reaction time (hr) | FFA | MAG | DAG | TAG | Glycerol |
| 0 | 90.2 | | | | 9.8 |
| 8 | 6.2 | 0.04 | 5.5 | 87.8 | not measured |
| 22 | 4.6 | 0.01 | 1.0 | 94.3 | not measured |

When operating at 70° C., for 22 hours, a condensation triacylglycerol oil having a high TAG level and low DAG and MAG levels was obtained. The product mixture was similar to average traded crude palm oil in FFA and TAG contents and the levels of DAG and MAG were very low. The very low levels of MAG obviates the need for molecular distillation of the condensation triacylglycerol oil and thus produces a product ready for use without further heat processing.

### Example 2

### Condensation of Oleic Acid and Glycerol at 80° C.

Free fatty acids and glycerol (in a 3.1:1 molar ratio) were incubated with a lipase enzyme. Oleic acid, 50 grams, was stirred with 2, 6 or 10 wt. % LIPOZYME® 435 at 80° C. Subsequently, 5.3 grams of glycerol was added dropwise over 10 minutes and the reaction mixture was stirred at 200 rpm for 48 hours at 80° C. under reduced pressure (0.8-2.13 kPa). Quantification of triacylglycerols, diacylglycerols, and monoacylglycerols and free fatty acids was performed using HPLC-SEC. The results are shown in Table 2.

TABLE 2

| Enz. dosage | Reaction time | Composition (wt. %) | | | |
|---|---|---|---|---|---|
| (wt. % of oil) | (hours) | TAG | DAG | MAG | FFA |
| 2 | 14 | 83.8 | 8.5 | 0.0 | 7.7 |
| 2 | 18 | 88.7 | 4.2 | 0.1 | 6.9 |
| 6 | 8 | 85.9 | 6.6 | 0.2 | 7.3 |
| 6 | 12 | 84.6 | 7.6 | 0.4 | 7.4 |
| 6 | 14 | 86.6 | 6.7 | 0.3 | 6.3 |
| 6 | 18 | 92.5 | 2.6 | 0.0 | 4.9 |
| 10 | 8 | 87.1 | 5.5 | 0.0 | 7.3 |
| 10 | 10 | 86.0 | 6.9 | 0.0 | 7.1 |
| 10 | 12 | 87.3 | 5.6 | 0.3 | 6.8 |
| 10 | 14 | 89.0 | 4.8 | 0.2 | 6.0 |
| 10 | 18 | 92.4 | 2.3 | 0.0 | 5.3 |

Condensation triacylglycerol oils low in monoacylglycerol (below 0.4 wt. %), very rich in triacylglycerol (86-93 wt. %) and with maximal 7.7 wt. % unreacted free fatty acids were obtained after 8-14 hours reaction.

### Example 3

### Deodorization of Condensation Triacylglycerol Oil

A mixture of free fatty acids reflecting the fatty acid composition of an exemplary palm free fatty acid feedstock comprising saturated and monounsaturated fatty acids comprising 80.6 wt. % unsaturated fatty acids was prepared. Palmitic acid (120 grams), stearic acid (50 grams), oleic acid (710 grams) and linoleic acid (130 grams) were mixed. The fatty acid mixture (376 grams) was heated to 50° C. and LIPOZYME® 435 lipase (37.6 grams) was added; these were stirred at 200 rpm for 10 minutes at 50° C. Glycerol (40.8 grams) was added with stirring at 450 rpm and the condensation reaction was carried out for 22 hours at 50° C. under reduced pressure (0.66 kPa) to eliminate water formed in the condensation reaction from the reaction mixture. The reaction progress was monitored by measuring the content of FFA in the reaction mixture. After the reaction, the enzyme was separated from the condensation triacylglycerol oil by filtration. The condensation triacylglycerol oil (200 grams) was deodorized by heating to at 265° C. for 45 minutes under vacuum (0.3 kPa) with sparge steam provided by allowing water to be drawn into the hot oil. The total amount of sparge steam was 5 wt. % of the weight of oil, provided throughout the deodorization process. The results are shown in Table 3.

TABLE 3

| Reaction time | FFA |
|---|---|
| (hr) | (wt. %) |
| 0.5 | 79.9 |
| 1 | 58.4 |
| 2 | 39.9 |
| 3 | 33.6 |
| 6 | 24.9 |
| 22 | 7.2 |
| After | 0.72 |
| deodorization | |

A triacylglycerol product enriched in monounsaturated fatty acids reflecting the fatty acid composition of an exemplary palm triacylglycerol product comprising 80.6 wt. % unsaturated fatty acids was obtained.

### Example 4

### Deodorization of Condensation Product Mixtures

Fatty acid feedstock comprising saturated and monounsaturated fatty acids from Example 3 (376 grams) was heated to 50° C. and LIPOZYME® 435 lipase (37.6 grams) was added these were stirred at 200 rpm for 10 minutes at 50° C. Glycerol (40.8 grams) was added with stirring at 450 rpm and the condensation reaction was carried out for 22 hours at 50° C. under reduced pressure (0.66 kPa) to eliminate water formed in the condensation reaction from the reaction mixture to produce a product containing 7.2 wt. % FFA. The reaction progress was monitored by measuring the content of FFA in the reaction mixture. After the reaction, the enzyme was separated from the condensation triacylglycerol oil by filtration. Half of this product was set aside. The recovered enzyme was mixed with a second aliquot of the fatty acid mixture (376 grams) and glycerol (40.8 grams) and the condensation reaction was repeated for 22 hours. A second lot of product containing 7.2 wt. % FFA was obtained. The portion of the first product that had been set aside was combined with the second product and subjected to deodorization deodorized by heating to at 265° C. for 75 minutes under vacuum (0.3 kPar) with sparge steam provided by allowing water to be drawn into the hot oil. The total amount of sparge steam was 5% of the weight of oil, provided throughout the deodorization process. The properties were measured using standard AOCS (American Oil Chemists' Society) methods which are widely available. The free fatty acid content of the condensation triacylglycerol oil mixtures during deodorization and properties of the deodorized condensation triacylglycerol oil are given in Table 4.

TABLE 4

| Deodori- | | | |
|---|---|---|---|
| zation | | Value | |
| Time | Analyte | (wt. %) | Measurement method |
| 30 min. | FFA (wt. %) | 2.24 | AOCS Ca 5a-40 |
| 45 min. | FFA (wt. %) | 0.72 | AOCS Ca 5a-40 |
| 60 min | FFA (wt. %) | 0.025 | AOCS Ca 5a-40 |
| 75 min. | FFA (wt. %) | 0.016 | AOCS Ca 5a-40 |

After deodorization

| | | | |
|---|---|---|---|
| 75 min. | TAG (wt. %) | 96.6 | Gas chromatography |
| 75 min. | DAG (wt. %) | 3.3 | Gas chromatography |
| 75 min. | MAG (wt. %) | 0.01 | Gas chromatography |
| 75 min. | Color (red) | 1.0 | AOCS Cc 13j-97 |
| 75 min. | Color (yellow) | 5.0 | AOCS Cc 13j-97 |
| 75 min. | Flavor | Good, | AOCS Cg 2-83 |
| | | mild, 9 | |
| 75 min. | Mettler Dropping Point | 81.9° F./ | AOCS Cc 18-80 |
| | | 27.7° C. | |
| 75 min. | OSI | 2.3 | AOCS Cd 125-92 |
| 75 min. | Solid fat content 10° C. | 17.4 | Cd 16b-93 |
| 75 min. | Solid fat content 21.1° C. | 3.9 | Cd 16b-93 |
| 75 min. | Solid fat content 26.7° C. | 1.6 | Cd 16b-93 |
| 75 min. | Solid fat content 33.3° C. | 0.2 | Cd 16b-93 |
| 75 min. | Solid fat content 40° C. | 0 | Cd 16b-93 |

The condensation triacylglycerol oil required a higher deodorization temperature and longer deodorization time than typical vegetable oil because of the initial presence of higher levels of FFA in the condensation triacylglycerol oil. A triacylglycerol product enriched in monounsaturated fatty acids reflecting the fatty acid composition of an exemplary palm triacylglycerol product comprising 80.6 wt. % unsaturated fatty acids was obtained. The deodorized condensation triacylglycerol oil was low in FFA and MAG and had good flavor, color, dropping point, and solid fat content properties.

### Example 5

### Condensation of Glycerol and Free Fatty Acids to Form Triacylglycerols at 80° C. Under 0.66 kPa Vacuum

Linoleic acid (93.3 grams) was condensed with glycerol (10.2 grams) using LIPOZYME® 435 lipase (9.3 grams) under reduced pressure (0.66 kPa) to eliminate water formed in the condensation reaction from the reaction mixture. After the reaction the enzyme was recovered by filtration and contacted with fresh substrate solution. The reaction progress was monitored by measuring the content of FFA in the reaction mixture. The results are shown in Table 5.

TABLE 5

| | FFA (wt. %) | | |
|---|---|---|---|
| Reaction time (hr) | First use | Second use | Third use |
| 1 | 27.2 | 29.5 | 23.5 |
| 2 | 16.9 | 19.9 | 4.4 |
| 3 | 8.6 | 15.4 | 9.5 |
| 4 | 5.7 | 11.5 | 6.6 |
| 6 | 4.2 | 8.6 | 4.6 |
| 8 | 4.0 | 8.6 | No data |

After three uses of the enzyme at 80° C. to produce condensation triacylglycerol oil, no detectable activity loss took place.

### Example 6

### Condensation of Glycerol and Palm Fatty Acid Distillate to Form Triacylglycerols

A palm free fatty acid feedstock comprising saturated and monounsaturated fatty acids (palm fatty acid distillate, PFAD) from physical refining of palm oil was subjected to distillation in a short-path still (VTA-USA, Rock Hill, S.C., USA) under conditions suitable for fractionation to distill off palmitic acid as distillate. It was expected that compounds that could potentially decrease lipase activity would also be removed in the distillate. Palm fatty acid distillate (700 grams) was subjected to short path distillation carried out at 110° C. and vacuum (0.028 kPa). A feed rate of 5 mL/minute was used. The residue (577 grams) from distilled PFAD contained 77.5 wt. % FFA, 3.3 wt. % MAG, 8.4 wt. % DAG, 2.6 wt. % glycerol, and 4.7 wt. % TAG. Subsequently, a second batch of PFAD was subjected to short-path distillation in the same manner to provide a greater amount of residue from distilled PFAD; the residue from the second batch comprised 522 grams of residue from distilled PFAD.

The distillation process provided residues from distilled PFAD enriched in oleic acid and having a lower content of palmitic acid (Table 6A).

TABLE 6A

| | PFAD as | | Batch 1 | | Batch 2 |
|---|---|---|---|---|---|
| | received | Batch 1 | Distil- | Batch 2 | Distil- |
| Fatty acid | (wt. %) | Residue | late | Residue | late |
| Caprylic | 0.05 | 0.04 | 0.05 | 0.05 | 0.03 |
| Capric | 0.04 | 0.03 | 0.03 | 0.05 | 0.02 |
| Lauric | 0.34 | 0.24 | 0.69 | 0.26 | 0.47 |
| Myristic | 1.36 | 0.64 | 4.28 | 0.63 | 3.24 |
| Palmitic | 47.84 | 42.81 | 67.64 | 41.70 | 66.08 |
| Palmitoleic | 0.18 | 0.03 | 0.05 | 0.08 | 0.05 |
| Stearic | 3.97 | 4.54 | 1.63 | 4.62 | 1.88 |
| Oleic | 34.49 | 38.43 | 17.74 | 39.11 | 20.33 |
| Linoleic | 8.50 | 9.37 | 4.82 | 9.53 | 5.40 |
| Arachidic | 0.30 | 0.38 | 0.04 | 0.39 | 0.05 |
| Gadoleic | 0.11 | 0.14 | nd | 0.14 | nd |
| Linolenic | 0.28 | 0.31 | 0.17 | 0.31 | 0.22 |
| Behenic | 0.07 | 0.08 | 0.01 | 0.09 | nd |
| Total trans FA | 0.39 | 0.49 | 0.35 | 0.45 | 0.32 |
| Total C18:1 trans FA | 0.26 | 0.32 | 0.08 | 0.32 | 0.12 |
| Total C18:1 cis FA | 35.35 | 39.18 | 18.08 | 39.89 | 20.71 |
| Total C18:2 trans FA | 0.13 | 0.12 | 0.20 | 0.10 | 0.17 |
| Total C18 2 cis FA | 8.50 | 9.37 | 4.82 | 9.53 | 5.40 |
| Total C18:3 trans FA | 0 | 0.04 | 0.07 | 0.03 | 0.04 |
| Total C18:3cis FA | 0.28 | 0.31 | 0.17 | 0.31 | 0.22 |
| CLA Region | 0.02 | 0.04 | 0.02 | 0.04 | 0.00 |
| Total Saturated FAs | 54.10 | 48.96 | 74.78 | 48.02 | 72.13 |
| Total C18:0 FA | 3.97 | 4.54 | 1.63 | 4.62 | 1.88 |
| Total C16:0 FA | 47.84 | 42.81 | 67.64 | 41.70 | 66.08 |

The PFAD residue (94 grams) was subjected to condensation for 5 hours using LIPOZYME® 435 lipase (18 grams) at 82.5° C. and under reduced pressure (0.66 kPa) to eliminate water formed in the condensation reaction from the reaction mixture. The enzyme was contacted with the PFAD residue at 50° C. for ten minutes, then glycerol (10.2 grams) was added and the reaction was carried out for 5 hours. After the reaction, the LIPOZYME® 435 lipase was recovered and re-used for a total of six reactions (recovered five times). The reaction progress was monitored by measuring the content of FFA in the reaction mixture. The results are shown in Table 6B.

TABLE 6B

| | FFA (wt. %) | | | | | |
|---|---|---|---|---|---|---|
| Reaction | First | Second | Third | Fourth | Fifth | Sixth |
| time (hr) | use | use | use | use | use | use |
| 1 | 8.2 | 7.6 | 8.1 | 8.2 | 11.3 | 13.1 |
| 2 | 1.0 | 1.4 | 1.1 | 1.4 | 3.0 | No data |
| 3 | 0.5 | 0.6 | 0.4 | 0.4 | 1.0 | 1.5 |

Loss of enzyme activity in the production of condensation triacylglycerol oil was decreased by short-path distillation of the PFAD, and no activity loss was observed in the first four uses. Even though the fifth reaction experienced a temperature over-run to about 100° C. for about 10 minutes, little activity loss was observed. After the fifth use of the enzyme it was still capable of producing triacylglycerol oils having low contents of free fatty acids.

### Example 7

### Designed Experiment for Condensation of FFA with Glycerol to Form Condensation Triacylglycerol Oil

A designed experimental for optimizing the TAG condensation reaction from fatty acid was set up using variables of temperature (60° C.-90° C.) and reduced pressure (0.66-13.33 kPa) to eliminate water formed in the condensation reaction from the reaction mixture. Technical grade oleic acid was mixed with palmitic acid and stearic acid to produce a FFA mixture containing about 85 wt. % unsaturated fatty acids and 15 wt. % saturated fatty acids. Free fatty acid substrate (94 grams) was incubated with LIPOZYME® 435 lipase (9.4 grams), then with glycerol (10.2 grams) for 5 hours. The results are shown in Table 7.

TABLE 7

| Run | T | Vacuum | FFA | DAG | TAG |
|---|---|---|---|---|---|
| # | ° C. | (kPa) | wt. % | wt. % | wt. % |
| 1 | 75 | 13.33 | 29 | 20.5 | 37.3 |
| 2 | 75 | 7 | 18 | 15.1 | 62.3 |
| 3 | 75 | 7 | 14.1 | 17.7 | 63.7 |
| 4 | 60 | 0.67 | 14.6 | 17.1 | 64.1 |
| 5 | 75 | 7 | 17.5 | 13.2 | 65.3 |
| 6 | 90 | 7 | 11.3 | 14.4 | 71.8 |
| 7 | 60 | 13.33 | 31.8 | 25.2 | 25.1 |
| 8 | 60 | 7 | 33.2 | 18 | 36.5 |
| 9 | 75 | 7 | 12.4 | 16.3 | 67.7 |
| 10 | 90 | 0.67 | 5.6 | 4.2 | 90 |
| 11 | 75 | 7 | 15.5 | 19.9 | 58.3 |
| 12 | 75 | 0.67 | 6.6 | 10.2 | 82.3 |
| 13 | 90 | 13.33 | 15.2 | 14.5 | 66.4 |

The condensation of FFA to form condensation triacylglycerol oil depended upon vacuum and temperature. At high temperature, the vacuum effect was not as strong as at low temperature. The reaction was more favorable at higher temperature (up to 90° C.) and stronger vacuum. Stronger vacuum (0.66 kPa) was required to obtain lower DAG levels in the product mixture. The model predicted that a reaction time of 5-8 hours can be expected to produce condensation triacylglycerol oil having levels of DAG and FFA similar to crude palm oil, at 82.5° C., to provide a palm oil substitute triacylglycerol product enriched in monounsaturated fatty acids. The established model allowed prediction of the composition of the reaction mixture at different reaction conditions.

### Example 8

Confirmation of Projected Optimal Conditions for Condensation of FFA with Glycerol to Form Triacylglycerols

The fatty acid mixture reflecting the fatty acid composition of an exemplary palm free fatty acid feedstock comprising saturated and monounsaturated fatty acids used in Example 3 (94 grams) was incubated with LIPOZYME® 435 (9.4 grams) for 10 minutes with stirring at 50° C., then an approximately stoichiometric amount of glycerol (10.2 grams) was added. The reaction mixture was stirred at 82.5° C. under reduced pressure (0.66 kPa) to eliminate water formed in the condensation reaction from the reaction mixture for 7 hours and sampled at 5, 6, and 7 hours. The results are shown in Table 8.

TABLE 8

| | Composition (wt. %) | | | |
|---|---|---|---|---|
| Reaction time (hr) | FFA | MAG | DAG | TAG |
| 5 | 5.66 | 0.08 | 6.79 | 87.1 |
| 6 | 3.5 | 0.04 | 4.18 | 92.1 |
| 7 | 2.6 | 0.02 | 2.53 | 94.8 |
| Crude palm oil Rule 6.7 | | | <5% | |
| Average traded crude | 3.5 (max. 5) | 0.2 | 4-8 | <98 |
| palm oil (from Table A) | | | | |

After 6 hours, a palm oil substitute triacylglycerol product enriched in monounsaturated fatty acids comprising enzymatically condensed palm oil that is similar in TAG, DAG, MAG and FFA content to average traded crude palm oil was produced in a single processing step. At 7 hours, the TAG content was even higher, and very low levels of DAG, MAG and FFA were obtained. The very low levels of MAG obviates the need for molecular distillation of the condensation triacylglycerol oil and thus produces a product ready for use without further heat processing.

### Example 9

### Enrichment of Unsaturated Fatty Acids from Palm Olein Free Fatty Acids and Production of Palm-Source Oil Low in Saturated Fatty Acids

Palm olein free fatty acid feedstock (PFA) comprising saturated and monounsaturated fatty acids (split palm olein fatty acids, 1041 liters, PMC Biogenix, Memphis, Tenn., USA) was subjected to separation by distillation at 225° C. and a reflux ratio targeted at 3 to obtain a first run residue fraction (about 790 liters) depleted in palmitic acid and enriched in oleic acid and linoleic acid. The distillate obtained was about 189 liters. In a second run, PFA feed was distilled in the same manner to obtain about 1020 liters of a second run residue depleted in palmitic acid and enriched in oleic acid and linoleic acid and about 720 liters of distillate. The composition of the PFA feed and two distillation residues are shown in Table 9A.

TABLE 9A

| | | | First run | Second run |
|---|---|---|---|---|
| | | | residue of | residue of |
| | | PFA as | PFA distilled | PFA distilled |
| | | received | at 125° C. | at 125° C. |
| Fatty acid | Abbreviation | (wt. %) | (wt. %) | (wt. %) |
| Myristic | C14:0 | 1.00 | 0.12 | 0.14 |
| Palmitic | C16:0 | 40.10 | 22.98 | 10.17 |
| Stearic | C18:0 | 4.43 | 5.94 | 7.33 |
| Oleic | C18:1n9 cis | 40.57 | 53.74 | 62.32 |
| Linoleic | C18:2n6 cis | 10.59 | 13.91 | 15.64 |
| Linolenic | C18:3n3cis | 0.23 | 0.45 | 0.26 |
| Total trans FA | | 0.56 | 0.78 | 1.18 |
| Total C18:1 cis FA | | 41.39 | 54.72 | 63.66 |
| Total C18 2 cis FA | | 10.59 | 13.92 | 15.65 |
| Total C18:3 cis FA | | 0.23 | 0.45 | 0.26 |
| Total Saturated FAs | | 46.52 | 29.86 | 18.69 |

Residues enriched in free monounsaturated fatty acid (oleic acid) were obtained. The palm-sourced residue fractions enriched in free monounsaturated fatty acids were suitable for esterifying with glycerol to obtain a condensation triacylglycerol that is similar in triacylglycerol, diacylglycerol, monoacylglycerol, and free fatty acid content to average traded crude palm oil, wherein fatty acids used in the condensation are source from palm oil. More exhaustive distillation of palm-sourced free fatty acids would provide a residue further depleted in palmitic acid and enriched in oleic acid (80 wt. % oleic acid).

The first run residue enriched in free monounsaturated fatty acids from the PFA distilled at 225° C. (133 kg) was mixed with glycerol (14.5 kg) to form a reaction mixture in a vessel heated to maintain the temperature of the vessel contents at 82.5° C. and stirred adequately to maintain a dispersion of glycerol in FFA. A column of lipase was prepared by packing Novozymes NS-40083 lipase (18 kg) in a heated reactor column (diameter=23 cm; bed height=61 cm), then filling the column with the first run residue enriched in free monounsaturated fatty acids (residue) preheated to 50° C. The column was heated to maintain the temperature of the contents of the column at 80° C. and the mixture of first run residue and glycerol was passed through the lipase column at 113.6 liters/minute to esterify glycerol and the first run residue enriched in free monounsaturated fatty acids. The reaction mixture passing out of the column was subjected to vacuum (0.66 kPa) to eliminate water formed in the condensation reaction from the reaction mixture by spraying the reaction mixture into a vessel held under vacuum to obtain dewatered reaction mixture. The dewatered reaction mixture was cycled back through the lipase column, then cycled to. The cycling of partially reacted substrate was continued for 22 hours until a condensation triacylglycerol product enriched in monounsaturated fatty acids comprising 87.22 wt. % TAG, 2.56 wt. % DAG, 0.13 wt. % MAG, and 6.53 wt. % FFA was obtained. The condensation triacylglycerol product enriched in monounsaturated fatty acids was deodorized at 220-240° C. to yield a palm oil substitute triacylglycerol product enriched in monounsaturated fatty acids comprising 94.8 wt. % TAG, 5.02 wt. % DAG, 0.05 wt. % MAG, and 0.105 wt. % FFA. The palm oil substitute triacylglycerol product contained 55.3% oleic acid.

The second run residue enriched in free monounsaturated fatty acids from PFA distilled at 225° C. was esterified with glycerol and deodorized substantially as the first run. After deodorizing, a second palm oil substitute triacylglycerol product enriched in monounsaturated fatty acids comprising 95.3 wt. % TAG, 4.61 wt. % DAG, 0.02 wt. % MAG, and 0.05 wt. % FFA was obtained. The palm oil substitute triacylglycerol product enriched in monounsaturated fatty acids contained 62.9% oleic acid.

In a third and fourth run, palm olein free fatty acid feedstock (1220 kg, PMC Biogenix, Memphis, Tenn.) was distilled to yield a third run residue and a fourth run residue. The composition of the PFA feed and two distillation residues are shown in Table 9B.

TABLE 9B

| | PFA as | Third run residue | Fourth run residue |
|---|---|---|---|
| | received | of PFAD | of PFAD |
| Fatty acid | (wt. %) | distillation | distillation |
| Myristic | 1.01 | 0.17 | 0.07 |
| Palmitic | 40.04 | 22.97 | 10.00 |
| Stearic | 4.48 | 5.93 | 7.28 |
| Oleic | 40.85 | 53.47 | 63.62 |
| Linoleic | 10.56 | 13.62 | 15.47 |
| Linolenic | 0.18 | 0.29 | 0.24 |
| Total trans FA | 0.49 | 1.56 | 1.16 |
| Total C18:1 cis FA | 41.57 | 54.52 | 64.80 |
| Total C18 2 cis FA | 10.56 | 13.62 | 15.47 |
| Total C18:3 cis FA | 0.21 | 0.29 | 0.24 |
| Total Saturated FAs | 46.66 | 29.65 | 17.89 |

The third and fourth run residues were separately combined with glycerol and separately esterified with glycerol substantially as the first and second run except that a second lipase column of similar capacity was added in parallel to the first enzyme reactor. The resulting palm oil substitute triacylglycerol products enriched in monounsaturated fatty acids were deodorized to yield a third run palm oil substitute triacylglycerol product enriched in monounsaturated fatty acids comprising 95.3 wt. % TAG, 4.41 wt. % DAG, less than 0.01 wt. % MAG, and 0.024 wt. % FFA and containing 52.3 wt. % oleic acid; the fourth run palm oil substitute triacylglycerol product enriched in monounsaturated fatty acids comprised 89.3 wt. % TAG, 6.2 wt. % DAG, 0.01 wt. % MAG, and 0.07 wt. % FFA and 64.0 wt. % oleic acid.

### Example 10

### Enrichment of Unsaturated Fatty Acids from Palm Olein Free Fatty Acids and Production of Palm-Source Oil Low in Saturated Fatty Acids

Palm free fatty acid feedstock (PFA) comprising saturated and monounsaturated fatty acids (split palm oil fatty acids, Wilmar, Shanghai, China) were distilled substantially as outlined in Example 9 to obtain a fifth run residue fraction depleted in palmitic acid and enriched in oleic acid and linoleic acid. The fifth run residue was split into a first lot (135 kg) and a second lot (116 kg). Each lot was mixed with a stoichiometric amount of glycerol. A single column of lipase was prepared by packing Novozymes 435 lipase (3.2 KG) and carrying out the condensation reaction of each lot substantially as outlined in Example 9. The condensation triacylglycerol product enriched in monounsaturated fatty acids lots were mixed and deodorized at 220-240° C. to yield a palm oil substitute triacylglycerol product enriched in monounsaturated fatty acids comprising 96.0 wt. % TAG, 3.9 wt. % DAG, 0.02 wt. % MAG, and 0.08 wt. % FFA. The palm oil substitute triacylglycerol product contained 17.1% saturated fatty acids, 66.0% oleic acid, 12.2% linoleic acid, and 0.15% linolenic acid. The OSI (110° C.) value of the palm oil substitute triacylglycerol product was 4 hours, and the peroxide vaue was 0. When the palm oil substitute triacylglycerol product enriched in monounsaturated fatty acids was supplemented with 15 ppm citric acid and 800 ppm mixed tocopherols, the OSI time was 20.1 hours.

The palm oil substitute triacylglycerol product enriched in monounsaturated fatty acids with 15 ppm Citric Acid and 800 ppm mixed tocopherols was subjected to a frying test in 6.8 kg bench top fryers. Oil was heated to 188° C. (370° F.) for 8 hours/day for 10 days. Twenty batches of cubed potato samples (230 grams/batch, Freezerfridge 12 mm (½ inch) Hash Brown Cube, Simplot, Boise, Id., USA) were fried every day. Eight hundred grams of palm oil substitute triacylglycerol product was added to the fryer each morning to replace oil lost. After 10 days of frying, the OSI value of the palm oil substitute triacylglycerol product was 26.1 hours. The fried potato cubes were blind-tasted by experienced oil quality tasters on the mornings of days 1, 3, 5, 6, 8 and 10. Both the flavor and frying performance of the palm oil substitute triacylglycerol product were, and were comparable to palm olein.

### Example 11

Production of Palm-Source Oil from Commercial Distilled Palm Oil Fatty Acids

A palm free fatty acid feedstock comprising distilled saturated and monounsaturated fatty acids was esterified with glycerol. According to the supplier (YiHai (Lianyungang) Oleochemical Ind. Co., Ltd), the distilled palm oil fatty acids (Product Code DP-1601) had the composition and characteristics shown in Table 11.

TABLE 11

| Fatty acid | Amount (% by GC) |
|---|---|
| Lauric | 0.07 |
| Myristic | 0.37 |
| Palmitic | 44.55 |
| Stearic | 7.01 |
| Oleic | 38.84 |
| Linoleic | 8.22 |
| Others | 0.94 |
| Acid value | 206.88 |
| Iodine value | 52.21 |
| Color | 0.2 Red, 1.0 Yellow |

This palm free fatty acid feedstock comprising saturated and monounsaturated fatty acids (300 grams) was condensed with glycerol (30.6 grams) using 9.3 grams of LIPOZYME® 435 lipase, as described in Example 7, at 82.5° C. under reduced pressure (0.66 kPa) with stirring at 450 rpm for 6 hours. After condensation, the condensed triacylglycerol product was deodorized for 30 minutes at 260° C. as described in Example 3 to yield a condensed triacylglycerol oil similar to palm oil, and comprising 96.17 wt. % TAG, 1.27 wt. % DAG, no MAG, and 0.19 wt. % FFA.

The LIPOZYME® 435 lipase was recovered after use and incubated again with distilled palm fatty acids (Product Code DP-1601) and glycerol at 82.5° C. under reduced pressure (0.66 kPa) with stirring at 450 rpm for 6 hours. After condensation, the condensed triacylglycerol product was deodorized for 30 minutes at 260° C. as outlined in Example 3 to yield a condensed triacylglycerol oil similar to palm oil, containing the fatty acid distribution shown in Table 11 and comprising 94.23 wt. % TAG, 3.19 wt. % DAG, no MAG, and 0.02 wt. % FFA.

### Example 12

### Production of Palm-Source Oil from Commercial Palmitic Acid

Distilled palmitic acid (98 wt. %) was obtained from YiHai (Lianyungang) Oleochemical Ind. Co., Ltd. The distilled free palmitic acid corresponds to a type of distillate fraction that can be obtained by the distillate outlined in Example 6. According to the supplier, the distilled palmitic acid had the composition and characteristics shown in Table 12.

TABLE 12

| Fatty acid | Amount (percent by GC) |
|---|---|
| Myristic | 1.19 |
| Palmitic | 98.08 |
| Others | 0.73 |
| Acid value | 219.65 |
| Iodine value | 0.09 |

Distilled palmitic acid (300 grams) was condensed with glycerol (30.6 grams) with 9.3 grams of LIPOZYME® 435 lipase, as described in Example 7, at 82.5° C. under reduced pressure (0.66 kPa) with stirring at 450 rpm for 6 hours. After condensation, the condensed triacylglycerol product was deodorized for 30 minutes at 260° C. as described in Example 3 to yield a condensed triacylglycerol oil comprising 95.43 wt. % TAG, 2.22 wt. % DAG, no MAG, and 0.06 wt. % FFA.

### Example 13

### Test of Nitrogen Bubbling and Reduced Pressure for Removal of Water Generated in the Condensation Reaction

Free fatty acids and glycerol (in a 3.1:1 molar ratio) were incubated with a lipase enzyme in a batch system either under reduced pressure or sparged with nitrogen from the bottom of the reactor.

Oleic Acid (50 grams) was mixed with 2.5 g LIPOZYME® 435 lipase at 80° C. Subsequently, 5.3 grams glycerol was added drop wise over 10 minutes and the reaction mixture was stirred for 24 hours. The reduced pressure treatment was performed in conical flasks and stirring at 200 rpm for 24 hours at reduced pressure (0.45-1.2 kPa). The N₂-bubbling treatment was performed in a glass column (25 mm×218 mm). N₂-sparging (20-25 l/h) was applied from the bottom of the glass column for 24 hours. Product composition was determined by HPLC-SEC. Water content was determined by Coulometric Karl Fischer titration. The results are shown in Table 13.

TABLE 13

| | | Composition (wt. %) | | | | |
|---|---|---|---|---|---|---|
| Reaction | | | | | | Water |
| time | Process | | | | | content |
| (hours) | design | TAG | DAG | MAG | FFA | (ppm) |
| 0 | Reduced | — | — | — | 100 | 108 |
| 2 | pressure | 43.4 | 37.6 | 1.2 | 17.8 | 339 |
| 4 | | 77.5 | 15.4 | 0.3 | 6.8 | 96 |
| 6 | | 88.6 | 8.1 | 0.0 | 3.4 | 31 |
| 24 | | 94.0 | 4.3 | 0.0 | 1.6 | 0 |
| 0 | Nitrogen | — | — | — | 100 | 681 |
| 2 | bubbling | 23.9 | 49.1 | 3.4 | 23.6 | 635 |
| 4 | | 61.1 | 28.1 | 1.2 | 9.6 | 288 |
| 6 | | 85.0 | 13.2 | 0.3 | 1.5 | 90 |
| 24 | | 90.0 | 9.9 | 0.1 | 0.0 | 7 |

A condensation triacylglycerol oil comprising predominantly DAG and TAG was obtained after 6 hours. Both treatments (reduced pressure and N₂-bubbling) were able to remove released water and very limited water was accumulated during reaction (max 635 ppm water). Reduced pressure was most efficient in fast water removal and pushing the reaction towards high triacylglycerol formation. After 24 hours under reduced pressure, a palm oil substitute triacylglycerol product enriched in monounsaturated fatty acids was obtained.

### Example 14

### Lifetime of LIPOZYME™ 435 in the N₂-Bubbling Batch System

In a condensation (esterification) reaction, free fatty acids and glycerol (in a 3.1:1 molar ratio) were incubated with a lipase in a N2-bubbling system. Oleic Acid (50 grams) were mixed with 1 wt. % LIPOZYME® 435 lipase (0.5 grams) in a glass column (25 mm×218 mm), incubated at 80° C. and sparged with N₂ (˜20 l/h) from the bottom of the glass column. Subsequently, glycerol (5.3 grams) was added drop wise over 10 min and the mixture was reacted for 22 hours with continuous N₂-bubbling to ensure good mixing and facilitate removal of formed water. After 22 hours reaction, the ‘spent oil/product mixture’ was decanted off and a fresh portion oil+glycerol were added for next cycle. The enzyme was re-used 26 times equal to processing 2.6 tons oleic acid per kg enzyme. The results are shown in Table 14 and are based on average values from two replicated trials.

TABLE 14

| Cycle no | 2 | 6 | 10 | 14 | 18 | 22 | 26 |
|---|---|---|---|---|---|---|---|
| ton FFA/kg | 0.2 | 0.6 | 1 | 1.4 | 1.8 | 2.2 | 2.6 |
| LIPOZYME ® 435 | | | | | | | |
| TAG | 68 | 56 | 54 | 47 | 43 | 38 | 39 |
| DAG | 18 | 27 | 29 | 32 | 35 | 37 | 37 |
| MAG | 0 | 1 | 2 | 2 | 2 | 3 | 3 |
| FFA | 13 | 15 | 16 | 19 | 20 | 21 | 21 |
| SUM | 100 | 100 | 100 | 100 | 100 | 100 | 100 |

After 26 uses, the enzyme retained 57% of the original enzyme activity.

1. A process comprising:
a) providing a palm free fatty acid feedstock comprising saturated and monounsaturated fatty acids,
b) subjecting the feedstock to separation to obtain a fraction enriched in monounsaturated free fatty acids and a fraction enriched in saturated free fatty acids,
c) esterifying glycerol and the fraction enriched in monounsaturated fatty acids to produce a condensation triacylglycerol oil comprising a palm oil substitute triacylglycerol product enriched in monounsaturated fatty acids relative to palm oil,
wherein the esterification step c) is performed by contacting the glycerol and the fraction enriched in monounsaturated free fatty acids with one or more lipase enzymes under conditions comprising that water formed in the condensation reaction is eliminated from the reaction mixture; and,
a temperature of at least 70° C.

2. The process according to claim 1 wherein the esterification step c) is performed under conditions comprising a water content of less than or equal to 700 ppm, 650 ppm, less than or equal to 600 ppm, less than or equal to 550 ppm, or less than or equal to 500 ppm.

3. The process according to claim 1 wherein the esterification step c) is performed under conditions comprising a pressure of less than or equal to 40 kPa, less than or equal to 30 kPa, less than or equal to 20 kPa, less than or equal to 15 kPa, less than or equal to 10 kPa, less than or equal to 5 kPa, less than or equal to 4 kPa, less than or equal to 3 kPa, less than or equal to 2 kPa, less than or equal to 1 kPa.

4. The process according to claim 1 wherein the esterification step c) is performed under conditions comprising a temperature of at least 75° C., at least 80° C., at least 81° C., or at least 82° C.

5. The process according to claim 1 wherein the free fatty acid feedstock is derived from one or more oil selected from the group consisting of palm oil, palm-kernel oil, crude palm oil, refined palm oil, physically refined palm oil, deodorized palm oil, palm fractions, palm olein, palm stearin, palm mid fraction, and combinations of any thereof.

6. The process according to claim 1 wherein the free fatty acid feedstock is one or more oil selected from the group consisting of palm sludge oil, palm oil mill effluent, palm fatty acid distillate, and combinations of any thereof.

7. The process according to claim 1 wherein the free fatty acid feedstock is derived from fat splitting of one or more acylglycerols.

8. The process according to claim 1 wherein the free fatty acid feedstock is derived from enzymatic hydrolysis of one or more triacylglycerols.

9. The process according to claim 1 wherein separating the palm free fatty acid feedstock is conducted with a method selected from the group consisting of distillation, crystallization, centrifugation, urea precipitation, membrane filtration, molecular sieve, directed interesterification, and combinations of any thereof.

10. The process according to claim 1 wherein the fraction enriched in monounsaturated free fatty acids comprises at least 50 wt. %, at least 60 wt. %, at least 70 wt. %, at least 80 wt. %, at least 85 wt. %, or at least 90 wt. % monounsaturated free fatty acids.

11. The process according to claim 1 wherein the esterification in step c) is catalyzed by an immobilized lipase, preferably a lipase having at least 70%, at least 80%, at least 90%, at least 95%, at least 96%, at least 97%, at least 98%, or at least 99% sequence identity to the sequence shown in SEQ ID NO:1 of PCT patent application WO2008065060.

12. The process according to claim 1 wherein the fraction enriched in saturated free fatty acids comprises at least 65 wt. % saturated fatty acids and at least 60 wt. % palmitic acid.

13. The process according to claim 1 wherein the triacylglycerol product enriched in monounsaturated fatty acids relative to palm oil following step (c) has a content of monoacylglycerols less than or equal to 3 wt. %, less than or equal to 2 wt. %, less than or equal to 1.5 wt. %, less than or equal to 1 wt. %, or less than or equal to 0.5 wt. %.

14. The process according to claim 1 wherein the triacylglycerol product enriched in monounsaturated fatty acids relative to palm oil following step (c) has a content of diacylglycerols less than or equal to 8 wt. %, less than or equal to 7 wt. %, less than or equal to 6 wt. %, less than or equal to 5 wt. %, less than or equal to 4 wt. %, less than or equal to 3 wt. %, less than or equal to 2 wt. %, or less than or equal to 1 wt. %.

15. The process according to claim 1 wherein the triacylglycerol product enriched in monounsaturated fatty acids relative to palm oil following step (c) has a content of free fatty acids less than or equal to 3 wt. %, less than or equal to 2.5 wt. %, less than or equal to 2 wt. %, less than or equal to 1.5 wt. %, less than or equal to 1 wt. %, or less than or equal to 0.5 wt. %.

16. The process according to claim 1 wherein the triacylglycerol product enriched in monounsaturated fatty acids relative to palm oil comprises at least 90 wt. %, at least 91 wt. %, at least 92 wt. %, at least 93 wt. %, at least 94 wt. %, at least 95 wt. %, at least 96 wt. %, at least 97 wt. %, or least 98 wt. % triacylglycerol.

17. The process according to claim 1 wherein the triacylglycerol product enriched in monounsaturated fatty acids relative to palm oil comprises at least 65%, at least 70%, at least 75 wt. %, at least 80 wt. %, at least 85 wt. %, at least 90 wt. %, or at least 95 wt. % unsaturated fatty acids.

18. The process according to claim 1 comprising deodorization of the triacylglycerol product enriched in monounsaturated fatty acids relative to palm oil.

19. The process according to claim 1 where the saturated fatty ester fraction is subjected to esterification to provide a triacylglycerol.

20. The process according to claim 1 wherein the triacylglycerol product enriched in monounsaturated fatty acids relative to palm oil comprises 90 to 98 wt. % triacylglycerol, 4 to 8 wt. % diacylglycerol, a maximum of 0.2 wt. % monoacylglycerol, and a maximum of 5 wt. % free fatty acid.

21. A composition produced by the process of claim 1.
